(19) United States
(12) Patent Application Publication
(10) Pub. No.: US 2014/0261710 A1
(43) Pub. Date: Sep. 18, 2014

(54) Faucet Dryer

(71) Applicant: Sloan Valve Company, Franklin Park, IL (US)

(72) Inventor: Randall Gallob, Scottsdale, AZ (US)

(21) Appl. No.: 14/208,347

(22) Filed: Mar. 13, 2014

Related U.S. Application Data
(60) Provisional application No. 61/785,110, filed on Mar. 14, 2013.

(51) Int. Cl. E03C 1/04; A61L 2/16

(52) U.S. Cl. CPC E03C 1/04; A61L 2/16
USPC 137/1; 137/594

(57) ABSTRACT

A faucet with a forced air dryer. The forced air dryer provides forced air for drying an object adjacent the faucet water outlet.

### CROSS REFERENCE TO RELATED APPLICATIONS

This application claims priority to U.S. Provisional Application No. 61/785,110 filed Mar. 14, 2013, reference of which is hereby made in its entirety.

### FIELD OF THE INVENTION

The present invention generally relates water dispensers and dryers.

### BACKGROUND OF THE INVENTION

Water dispensing fittings such as faucets are ubiquitous in many buildings and public facilities. In particular, faucets are frequently utilized in kitchens and restrooms for hygiene. Individuals utilize the faucet for hand-washing. Typically it is desirable for hygienic, social, and practical reasons to dry one's hands after washing. For this reason towels or other physical objects that can be used to absorb water have traditionally been used, most commonly disposable paper towels.

Electronic Hand Dryers have also been utilized for removing the water from an individuals hand following a hand washing. A wall-mounted electronic dryer that blows warm air from a nozzle is a common sight in most commercial restrooms. However, the dryer must typically be positioned away from the sink, leaving the individual to travel a distance in the restroom with wet hands. Further, much of the energy of the dryer is wasted. There is a need for an automatic faucet and electronic hand dryer that provides for greater safety and hygiene with no water trail across the sink counter or floor and no additional water basin necessary.

### SUMMARY OF THE INVENTION

One embodiment of the invention relates to a faucet dryer system. The system comprises a faucet having a housing with a vanity opening and a dispensing end. A water line is disposed within the housing and connected to a water outlet positioned at the dispensing end of the housing. A forced air intake is positioned at the vanity opening end and a forced air outlet is positioned at the dispensing end of the housing. A forced air chamber is disposed within the housing and in communication with the forced air intake and the forced air outlet and disposed there between. A forced air system is in communication with the faucet, the forced air system having a forced air line attached to the faucet at the forced air intake.

Another embodiment relates to a fixture comprising a faucet having a housing with a vanity opening and a dispensing end. A water line is disposed within the housing and connected to a water outlet positioned at the dispensing end of the housing. A forced air intake is positioned at the vanity opening end and a forced air outlet is positioned at the dispensing end of the housing. A forced air chamber is disposed within the housing and in communication with the forced air intake and the forced air outlet and disposed there between.

Another embodiment relates to a method for sanitizing an object. A sanitizing cycle is initiated upon detecting the object. A forced air system is locked out and water dispensed from a water system having a water outlet. The water system is locked out to prevent further dispensing of water and forced air is dispensed from the forced air system through a forced air outlet adjacent the water outlet.

Additional features, advantages, and embodiments of the present disclosure may be set forth from consideration of the following detailed description, drawings, and claims. Moreover, it is to be understood that both the foregoing summary of the present disclosure and the following detailed description are exemplary and intended to provide further explanation without further limiting the scope of the present disclosure claimed.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1A is an illustration of one implementation of the proposed electronic faucet-dryer; FIG. 1B is an illustration of another implementation of the proposed faucet-dryer.

FIG. 2 illustrates a partial-cross sectional view of a faucet-dryer system positioned on a vanity.

FIG. 3A illustrates a first side view of a faucet of a faucet dryer system; FIG. 3B illustrates a second side view of a faucet of a faucet dryer system; FIG. 3C illustrates a front view of a faucet of a faucet dryer system; FIG. 3D illustrates a back view of a faucet of a faucet dryer system.

FIG. 4A is a cross-sectional view of the side view of FIG. 3B; FIG. 4B is front view of the faucet-dryer outlet; FIG. 4C is a top view of a faucet-dryer; FIG. 4D is a cross-sectional top view of the faucet-dryer of FIG. 4C.

FIG. 5A is a front view of a faucet-dryer system; FIG. 5B is a side view of a faucet-dryer system; FIG. 5C is a front view of a faucet-dryer outlet; FIG. 5D is a partially cut-away view of a faucet-dryer system positioned on a vanity.

FIG. 6 is a flow-chart depicting one mode of operation of a faucet-dryer system.

FIG. 7 illustrates and embodiment of a computer system of the present invention.

### DETAILED DESCRIPTION OF THE PREFERRED EMBODIMENTS

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

One implementation comprises a faucet-dryer system 100. The faucet-dryer system 100 may be positioned on a vanity or other “deck” 10. One implementation of the faucet-dryer system 100 comprises a faucet 200, a water system 300, a forced-air system 400, and a sensor system.

The faucet 200 includes a housing 210 having an inlet 220 and an outlet 230 with a passage defined there between. The faucet 200 may be presented with a housing 210 of various form factors as are known in the art. In one implementation the faucet 200 is positioned with the housing 210 extending above the deck 10 with the inlet 220 open to an area below the deck 10 and the outlet 230 positioned above a basin 11 of the deck 10. In one implementation, the faucet outlet 230 is positioned sufficiently above the basin 11 to accommodate a user's hands between the faucet 200 and the basin 11. In one implementation, the faucet above the deck is made of stainless steel.

The water system 300 is in communication with a water supply 30 and the faucet 200. In one implementation, the water system 300 includes a water line 330. The water line 330 is in communication with the water supply 30. For example, the water supply 30 may be a cold, i.e. not heated, water supply. A water supply valve 320 may be provided for controllably sealing the water line 330. The water supply valve 320 may be electronically controlled via a valve controller. It should be appreciated that the water supply valve 320 may comprise a valve, such as a solenoid or like mechanisms, for controlling flow of water through the water line 330.

The water line 330 extends into the faucet 200. The water line 330 receives water from the water supply 30 and channels it to faucet 200. In one implementation, the water supply 30 is positioned below the deck 10 and the water line 330 is positioned substantially below the deck 10 with a portion of the water line 330 entering the faucet 200 at the inlet 220 and extending through the passage in the faucet housing 210, such as an air chamber 213. The water line 330 may terminate prior to the faucet outlet 230, substantially at the faucet outlet 230 or prior to the faucet outlet 230 within the faucet 200. The water line 330 terminates at a water outlet 335. In one implementation, an aerator 338 is provided in the water line 330. For example, the aerator 338 is positioned adjacent the water outlet 335.

The forced air system 400 comprises an air blower 410 and a forced air line 430. The air blower 410 provides forced air. The forced air line 430 receives the forced air from the air blower 410 and channels to the forced air to the faucet 200. In one implementation, the air blower 410 is positioned below the deck 10 and the forced air line 430 is positioned substantially below the deck 10 with a portion of the forced air line 430 entering the faucet 200 at the inlet 220 and extending through the passage. The forced air line 430 may terminate prior to the faucet outlet 230, substantially at the faucet outlet 230 or prior to the faucet outlet 230 within the faucet 200. The forced air line 430 terminates at a forced air outlet 435. In one implementation the forced air system 400 includes a blower controller not shown.

The faucet 200 includes, in one implementation shown in FIG. 4a-4D a forced air intake 212. The forced air line 430 may connect to the housing 210 at the forced air intake 212. The housing 210 includes an air chamber 213 that defines a passage from the forced air intake 212 to the faucet outlet 230. In one implementation, the water line 330 is disposed substantially within the air chamber 213 to further facilitate heat exchange between the forced air and the water.

In one embodiment, the forced air system 400 further comprises a heater not shown. In one embodiment, the air blower 410 also includes the heater not shown. The forced air provided to the forced air line 430 is heated above room temperature by the heater (not shown). In one implementation, the air blower is a standard hand-drying device positioned to blow air upwards.

In one implementation, a heat exchanger 450 is provided. The heat exchanger 450 allows for the transfer of thermal energy between the forced air system 400 and the water system 300. For example, the heat exchanger may be positioned under the deck 10 and comprise a portion of the water line 330 coiled around the forced air line 430.

In one implementation, the heat exchanger allows the water provided by the water supply 30 to the water supply line 330 to be heated by utilizing the heat provided by the heater. Additionally, the material of the forced air line 430 and the water line 330 may be so selected as to maximize heat conduction from the forced air system 400 to the water system 300, thus allowing more thermal energy transfer to the 330 water line coiled around the forced air line 430.

In one implementation, the sensor system comprises a one sensor array 510. In one implementation the one sensor array 510 is positioned adjacent the faucet outlet 230 so as to have a zone of detection between the faucet outlet 230 and the basin 11. The at least one sensor array 510 utilizes presence detection sensors, for example active infrared sensors. The sensor array 510 includes, in one embodiment, at least one sensor (not shown) and at least one emitter (not shown).

The sensor system further comprises at least one sensor controller (not shown). The sensor array 510 is in communication with the controller (not shown), for example through sensor wires 560 or a wireless transmitter (not shown). In one implementation, the sensor controller, the water valve controller, and the blower controller are a single controller 240. In another implementation, the sensor controller, the water valve controller, and the blower controller are a separate controllers with the sensor controller in communication with the water valve controller and the blower controller.

A plurality of arrangements are possible for the relative numbers and positions of the water line outlet(s) 335 and the forced air line outlet(s) 435. In one implementation, the water line 330 and/or the forced air line 430 may be split into a multitude of water lines or forced air lines, respectively. In one implementation, two forced air line outlets 435 are provided on either side of a single water line outlet 335. In one implementation, one or more water lines 330 wrap around the forced air line 430 in the housing 210 to further facilitate heat exchange. In one implementation, a portion of the water line 330 is disposed within the forced air line 430 with the water line outlet 335 being disposed within the forced air line outlet 435.

In one implementation, a soap system is provided. The soap system provides for dispensing of soap from the faucet 200. The soap system includes a soap reservoir (not shown) and soap line (not shown). The soap reservoir for example, is a container for holding liquid soap. Alternatively, the soap reservoir may be a container for holding solid soap for interaction with water. In one implementation, the soap system further includes a pump (not shown) for providing motive force to the soap for dispensing. The soap reservoir and/or the pump are positioned below the deck 10 and the soap line is positioned substantially below the deck 10 with a portion of the soap line entering the faucet 200 at the inlet 220 and extending through the passage. The soap line may terminate prior to the faucet outlet 230, substantially at the faucet outlet 230 or prior to the faucet outlet 230 within the faucet 200. The soap line (not shown) terminates at a soap outlet (not shown). In one implementation the soap system includes a soap controller (not shown). The soap controller may, in one implementation, be the controller 240.

Various implementations provide for a more efficient hand washing process. In one embodiment, the heat exchanger 450 is capable of warming water for delivery from the faucet to about 90 degrees for about 7 seconds, which effectively saves or recovers 3 Kw of power, +30ΔT. There is a improved safety and hygiene with no water trail across a counter or floor and no additional water basin necessary; so there is no accumulated drops of water falling off of hands pooling onto the floor as users walk to a remotely mounted hand dryer. Further, the water provided from the faucet outlet 230 is warmed, providing user comfort, without additional energy requirements to heat the water or requiring a hot-water supply line. Further, certain implementations provide a single fixture for all hand washing necessities: soap, water and hand drying.

In one implementation, visual cues are provided, in part, through lighting. For example, a water dispensing visual cue 336, which may be a white LED and correspond generally to the location of the water outlet 335, is illuminated while the water is being dispensed. Likewise, a forced air dispensing visual cue 436, which may be a red LED correspond generally to the location of the forced air outlet 435, is illuminated while the forced air is being dispensed.

In one implementation two aerators are provided to define two water outlets at the terminal end of the faucet 200. Although various flows may be utilized for the water, in one example the water flows at 0.5 or 0.35 gpm. A slower flow rate will facilitate more thermal absorption by the water from the forced air system 400, not to mention a significant reduction in water consumption.

In one implementation, one sensor array 510 is provided to control initiation of a wash/dry cycle. For example, the user activates water for as long as user likes. The user can come in and out of sensor range turning water on and off until it reaches duration of 5 seconds, after 5 seconds in and out (soap wetting window) the water on/off cycle is locked out. After 5 seconds the user may stay in sensor range for as long as user requires water but once user breaks sensor range hand dryer will start automatically locking out water until the dryer cycle is completed by user. The forced air blower will start automatically after 3 second duration the sensor must take over control of hand dryer cycle. After hand drying cycle is complete controller resets to water stand by.

In one implementation, a plurality of sensor arrays 510 are provided. A water sensor array is in communication with the water supply system to provide water through the faucet to a user. A dryer sensor array is in communication with the water supply system to provide forced air through the faucet to a user.

In one implementation with the plurality of sensor arrays, a soap sensor array is in communication with a soap system to provide soap through the faucet to the user.

FIG. 6 illustrates one implementation of a method for controlling the faucet. At step 1001 the sensor array 510 detects a presence—typically a user's hand. The sensor array 510 provides a signal to the controller 240. The controller 240 activates the water supply valve 320 in response to receiving the signal from the sensor array 510 (1101). The water supply 30 provides water to the water line 330 for dispensing from the faucet outlet 230 (1102). The water continues to be dispensed for a predetermined time. For example, the water may be dispensed for 15 seconds. In one implementation, the water may be dispensed for the predetermined time or until the presence is no longer detected, whichever is shorter. As shown in FIG. 6, following the cessation of the water dispensing (1103), such as due to the predetermined time period, the system returns to a mode waiting for detection of a presence. If the sensor array 510 detects a presence (1001), the controller activates the forced air blower 410 to provide forced air through the forced air system 400 to the faucet outlet 230 (1202). In one implementation, the water system is locked out during operation of the forced air system (1201). The forced air continues to be dispensed for a predetermined time (1203). For example, the forced air may be dispensed for 15 seconds. In one implementation, the forced air may be dispensed for the predetermined time or until the presence is no longer detected, whichever is shorter (1204).

In one mode of operation of a faucet dryer having a water cycle and a dryer cycle, the user puts hands under housing 210 actuating water for as long as user likes. If water cycle is longer than 5 seconds, wave sensor will light up and be on stand by not allowing water to reactivate after water cycle is complete. If user touches or waves the hand, a forced-air sensor will blink a visual cue 436 and lock out water activating the forced air blower 410 for as long as user keeps hands under faucet 200. Once hand drying cycle is complete, the forced air sensor visual cue 436 goes off, the forced-air system is locked out and the faucet-dryer system goes into water system standby mode.

In one mode of operation of a faucet dryer having a water cycle and a dryer cycle and multiple sensor arrays 510, two separate electronic circuits are provided, one for the forced air blower 410 and one for the water supply valve 320 controlled by a single hand sensor, and actuated by two separate sensors. The sensor system includes a first sensor for detecting placement of the hands for washing and a second sensor for detecting the waving of hands for drying. Hands under the faucet 200 activates water flow for as long as sensor 510 detects hands. If the water cycle is longer than 5 seconds, the wave sensor will illuminate glowing red sensor, for example with “hand dryer” written on glowing sensor, for 5 seconds, putting hand dryer in stand by mode for 5 seconds. Hands under the faucet 200 activates the forced-air blower 410 for as long as sensor detects hands. Once the hand is removed from the sensor detection zone, the cycle is complete and the system 100 goes back to a hand detection mode.

In one implementation, the faucet/dryer system 100 also cleans and dries the basin 11 for the next user and warms the water for the next user giving 7 seconds of about 90 degree water. This is roughly a 35 degree temperature rise or delta T which equals around 3 KW of power and saving.

In one implementation, computer program logic, stored on a non-transient memory, is provided to provide visual and/or audio cues for a proper hand washing regime. For example, wetting hands, soaping hands, scrubbing hands for 15 seconds, rinsing hands, and drying hands all in one location; can be an option. In one implementation, with visual prompts, along with audible dryer prompts, a unique sequential method of self taught hand washing regime is achieved. Visual difference in appearance can allow for an obvious visual clue of a hand washing and drying device all in one package.

In one implementation a volume below the deck is physically separated and insulated to retain thermal energy, for example defining a volume below the deck that contains the water system 300 and the forced air system 400, that volume being insulated to aid warming the water from the water supply 30. Further, the housing 210 is, in one implementation, insulated to prevent transfer of heat to the exterior of the housing 210. In one implementation, temperature transducers (not shown) are included to cause dryer to activate if water line 330 temperature falls below a predetermined level, causing the dryer to run at low speed or some selected speed to raise water line 330 temperature.

In one implementation, override controls (not shown) are provided. For example, override controls are provided on the exterior of the housing 210 or on the top of the deck 10 to allow for easy deactivation of the dryer portion for routine restroom basin cleaning.

In one embodiment, shown in FIG. 7, a system 100 is provided. FIG. 7 shows an exemplary block diagram of an exemplary embodiment of a system 100 according to the present disclosure. For example, an exemplary procedure in accordance with the present disclosure can be performed by a processing arrangement 110 and/or a computing arrangement 110. Such processing/computing arrangement 110 can be, e.g., entirely or a part of, or include, but not limited to, a computer/processor that can include, e.g., one or more microprocessors, and use instructions stored on a computer-accessible medium (e.g., RAM, ROM, hard drive, or other storage device).

As shown in FIG. 7, e.g., a computer-accessible medium 120 (e.g., as described herein, a storage device such as a hard disk, floppy disk, memory stick, CD-ROM, RAM, ROM, etc., or a collection thereof) can be provided (e.g., in communication with the processing arrangement 110). The computer-accessible medium 120 may be a non-transitory computer-accessible medium. The computer-accessible medium 120 can contain executable instructions 130 thereon. In addition or alternatively, a storage arrangement 140 can be provided separately from the computer-accessible medium 120, which can provide the instructions to the processing arrangement 110 so as to configure the processing arrangement to execute certain exemplary procedures, processes and methods, as described herein, for example.

System 100 may also include a display or output device, an input device such as a key-board, mouse, touch screen or other input device, and may be connected to additional systems via a logical network. Many of the embodiments described herein may be practiced in a networked environment using logical connections to one or more remote computers having processors. Logical connections may include a local area network (LAN) and a wide area network (WAN) that are presented here by way of example and not limitation. Such networking environments are commonplace in office-wide or enterprise-wide computer networks, intranets and the Internet and may use a wide variety of different communication protocols. Those skilled in the art can appreciate that such network computing environments can typically encompass many types of computer system configurations, including personal computers, hand-held devices, multi-processor systems, microprocessor-based or programmable consumer electronics, network PCs, minicomputers, mainframe computers, and the like. Embodiments of the invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

Various embodiments are described in the general context of method steps, which may be implemented in one embodiment by a program product including computer-executable instructions, such as program code, executed by computers in networked environments. Generally, program modules include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps.

Software and web implementations of the present invention could be accomplished with standard programming techniques with rule based logic and other logic to accomplish the various database searching steps, correlation steps, comparison steps and decision steps. It should also be noted that the words “component” and “module,” as used herein and in the claims, are intended to encompass implementations using one or more lines of software code, and/or hardware implementations, and/or equipment for receiving manual inputs.

With respect to the use of substantially any plural and/or singular terms herein, those having skill in the art can translate from the plural to the singular and/or from the singular to the plural as is appropriate to the context and/or application. The various singular/plural permutations may be expressly set forth herein for the sake of clarity.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

We claim:

1. A faucet dryer system comprising:
a faucet having a housing with a vanity opening and a dispensing end;
a water line disposed within the housing and connected to a water outlet positioned at the dispensing end of the housing;
a forced air intake at the vanity opening end;
a forced air outlet positioned at the dispensing end of the housing;
a forced air chamber disposed within the housing and in communication with the forced air intake and the forced air outlet and disposed there between; and
a forced air system in communication with the faucet, the forced air system having a forced air line attached to the faucet at the forced air intake.

2. The system of claim 1, further comprising a presence sensor.

3. The system of claim 2, wherein the presence sensor is positioned at the dispensing end of the housing.

4. The system of claim 1 wherein a portion of a water line is disposed within the forced air line and the water outlet is disposed within the forced air outlet.

5. The system of claim 1, wherein the water line is disposed within the forced air chamber, the water line configured to exchange thermal energy with the air in the forced air chamber.

6. The system of claim 1, wherein the forced air system includes a blower and a heater.

7. The system of claim 6, further comprising a heat exchange wherein a water supply line connected to the water line is positioned in contact with the forced air supply line such that thermal energy is exchanged.

8. The system of claim 1, further comprising a soap dispensing system having a soap dispensing line disposed within the faucet and in communication with a soap dispenser positioned at the dispensing end.

9. The system of claim 1, wherein the water line and forced air system exchange of thermal energy heats water to about 90 degrees.

10. The system of claim 1, further comprising a plurality of lights associated with one or more of the water outlet and the forced air outlet.

11. The system of claim 1, further defining an insulated volume below a sink deck, the forced air system and a water supply system disposed within the insulated volume.

12. A fixture comprising:
a faucet having a housing with a vanity opening and a dispensing end;
a water line disposed within the housing and connected to a water outlet positioned at the dispensing end of the housing;
a forced air intake at the vanity opening end;
a forced air outlet positioned at the dispensing end of the housing; and
a forced air chamber disposed within the housing and in communication with the forced air intake and the forced air outlet and disposed there between.

13. The fixture of claim 12, further comprising a plurality of lights associated with one or more of the water outlet and the forced air outlet.

14. The fixture of claim 12, further comprising a presence sensor positioned at the dispensing end of the housing.

15. A method for sanitizing an object:
initiating a sanitizing cycle upon detecting the object;
locking out a forced air system and dispensing water from a water system having a water outlet;
locking out the water system to prevent further dispensing of water and dispensing forced air from the forced air system through a forced air outlet adjacent the water outlet.

16. The method of claim 15, wherein the lockout of the forced air system and dispensing of water corresponds to a water cycle time.

17. The method of claim 15, wherein the lockout of the water system and dispensing of forced air to a water cycle time.

18. The method of claim 15, further comprising exchanging thermal energy between the water system and the forced air system.

19. The method of claim 18, further comprising heating water dispensed from the water system to at least 90 degrees Fahrenheit.

20. The method of claim 18, further comprising maintaining water in a water supply line above a predetermined temperature.
